(19) United States
(12) Patent Application Publication
(10) Pub. No.: US 2024/0426810 A1
(43) Pub. Date: Dec. 26, 2024

(54) ANTI-PD1 THERAPY BASED ON RESPONSE TO IFN-I STIMULATION

(71) Applicant: UNIVERSITY HEALTH NETWORK, Toronto (CA)

(72) Inventors: David BROOKS, Toronto (CA); Giselle BOUKHALED, Toronto (CA); Heidi ELSAESSER, Toronto (CA)

(21) Appl. No.: 18/701,295

(22) PCT Filed: Oct. 14, 2022
(86) PCT No.: PCT/CA2022/051519

§ 371 Date: Apr. 15, 2024

Related U.S. Application Data
(60) Provisional application No. 63/256,104, filed on Oct. 15, 2021.

(51) Int. Cl. G01N 33/50; G01N 15/10; G01N 15/14; G01N 33/569

(52) U.S. Cl. CPC G01N 33/505; G01N 15/14; G01N 33/5023; G01N 33/56972; G01N 2015/1006; G01N 2333/56; G01N 2333/565; G01N 2333/57; G01N 2333/70503; G01N 2800/52

(57) ABSTRACT

There is described herein a method for predicting response to anti-PD1 based therapy in a subject with cancer, the method comprising: providing a sample of peripheral blood from the subject; adding an IFN-I to the sample; assessing T-cell response to IFN-I stimulation in the peripheral blood sample by measuring the expression of IFN-I stimulated genes; and predicting a better outcome in response to anti-PD1 therapy if the assessment in the previous step indicates T-cell resistance to IFN-I stimulation and predicting a poorer outcome in response to anti-PD1 therapy if the assessment step indicates T-cell responsiveness to IFN-I stimulation.

### FIELD OF THE INVENTION

The invention relates to cancer therapy and more particularly anti-PD1 therapy based response to IFN-I stimulation.

### BACKGROUND OF THE INVENTION

Type I IFNs (IFN-I; IFNα/β) are central regulators of anti-tumor immunity and responsiveness to immunotherapy, but also drive the feedback inhibition that underlies therapeutic resistance¹. IFN-Is activate, direct and sustain T cell function and differentiation both through intrinsic signaling and through modulation of antigen presenting cell (APC) integrity¹. Simultaneously, chronic IFN-I signaling induces the expression of inhibitory factors, including PDL1, IDO and IL-10, among others, that drives the functional T cell attenuation and suppressive differentiation programs (termed T cell exhaustion) that promote cancer escape^{1, 2, 3}. In conjunction, type II interferon (IFNγ) also enforces the expression of a similar profile of immunosuppressive molecules in the tumor microenvironment (TME)^{4, 5, 6, 7}, although as tumors start to favor an environment depleted of T cells (which are major producers of IFNγ) the maintenance of this immunosuppressive environment may be more promoted by IFN-I⁸. All IFN-Is signal through a dimeric IFN-I receptor (IFNR) that is expressed on all nucleated cells⁹. IFN-I signaling induces the expression of hundreds of IFN-I stimulated genes (ISGs) that have a broad range of functions^{1, 9}; and ultimately, it is the composition of these ISGs both in individual cells and at the population level that dictates their diverse effects and outcomes^{1, 10}. This diverse and to-date inseparable functionality has precluded their use as therapeutic targets.

### SUMMARY OF THE INVENTION

In an aspect, there is provided a method for predicting response to anti-PD1 based therapy in a subject with cancer, the method comprising: providing a sample of peripheral blood from the subject; adding an IFN-I to the sample; assessing T-cell response to IFN-I stimulation in the peripheral blood sample by measuring the expression of IFN-I stimulated genes; and predicting a better outcome in response to anti-PD1 therapy if the assessment in the previous step indicates lower T-cell response (i.e., resistance) to IFN-I stimulation and predicting a poorer outcome in response to anti-PD1 therapy if the assessment step indicates higher T-cell responsiveness to IFN-I stimulation.

In an aspect, there is provided a method of treating a subject with cancer, the method comprising administering to the subject a therapeutically effective amount of a PD1 inhibitor, wherein the subject had been determined to have a better outcome in response to anti-PD1 therapy using the methods described herein.

### BRIEF DESCRIPTION OF FIGURES

These and other features of the preferred embodiments of the invention will become more apparent in the following detailed description in which reference is made to the appended drawings wherein:

FIG. 1. CyTOF ISG panel validation. Contour plots showing unstimulated or IFNβ-stimulated expression of the indicated ISGs in CD45+ PBMCs from a representative healthy donor.

FIG. 2. Low IFN-I sensitivity is associated with longer overall survival in a cohort of melanoma patients treated with anti-PD1. Pre-therapy samples from 28 cutaneous melanoma patients were cultured overnight in the presence or absence of 1000 U/ml IFNβ. 22 lineage-defining markers were used for dimensionality reduction and clustering of ˜10×10⁶ cells. A. UMAP of 1×10⁶ randomly sampled events from the entire dataset. Shaded regions highlight specific T cell subsets of interest. Other cells are depicted in grey. B. Kaplan-Meier curves shown, comparing high and low IFN-I sensitivity score (ISS) in the indicated CD4 (left panels) and CD8 (right panels) T cell subsets. to indicates the number of patients in each group at the start of therapy. IFN-I sensitivity score (ISS) and IFN-I response capacity (IRC) are used interchangeably herein.

FIG. 3. High IDO induction by myeloid cells is associated with longer overall survival after anti-PD1 therapy. A. UMAP as in FIG. 2 highlighting myeloid cell phenotypes. Other cells are depicted in grey. B. Pearson correlation between myeloid cell ISS and PDL1 induction or IDO induction (left and middle panels) and correlation between PDL1 induction and IDO induction (right panels) across patient myeloid cells. CB, clinical benefit; NCB, no clinical benefit. C. Patients were stratified based on whether IDO induction was high or low. Kaplan-Meier curves shown, estimating overall survival in each group. to indicates the number of patients in each group at the start of therapy. D. Boxplot comparing the IDO induction of patients with high or low effector CD4 T cell ISS.

FIG. 4. Low IFN-I sensitivity in T cell subsets is associated with longer overall survival of lung cancer patients. 34 lung cancer patient pre-therapy PBMCs were analyzed as described previously. Kaplan-Meier curves comparing overall survival of patients stratified based on high or low ISS in the indicated T cell subsets.

FIG. 5. A Cox's proportional hazards model was developed that integrates the CD4 T cell IFN-I sensitivity, CD8 T cell IFN-I sensitivity and IDO induction by myeloid cells. The resulting formula was used to determine a risk score and then stratify patients into high or low risk groups. A. Receiver-operator characteristic curve plotting the true-positive and false-positive rates resulting from predicting 2-year survival for the datasets used to train and test the model. B. Kaplan-Meier curves comparing overall survival of patients stratified based on whether patients were predicted to be at high or low risk of progression.

FIG. 6. Kaplan-Meier curves comparing overall survival of patients stratified by whether they are at high or low risk of progression based on the model in FIG. 5, and whether they have high or low PDL1 expression in the tumor.

### DETAILED DESCRIPTION

In the following description, numerous specific details are set forth to provide a thorough understanding of the invention. However, it is understood that the invention may be practiced without these specific details.

Because of their fundamental role in almost all immune processes, a deeper understanding of IFN-Is complex functions could facilitate both new therapeutic targets to limit cancer growth and potentially be used as predictive biomarkers of response to a specific therapeutic modality. Thus, unravelling the biologic underpinnings of IFN-I responsive states and the complex coordination of IFN-I responses in immune cells prior to therapy may provide the biomarkers for predictive usage of immunotherapy

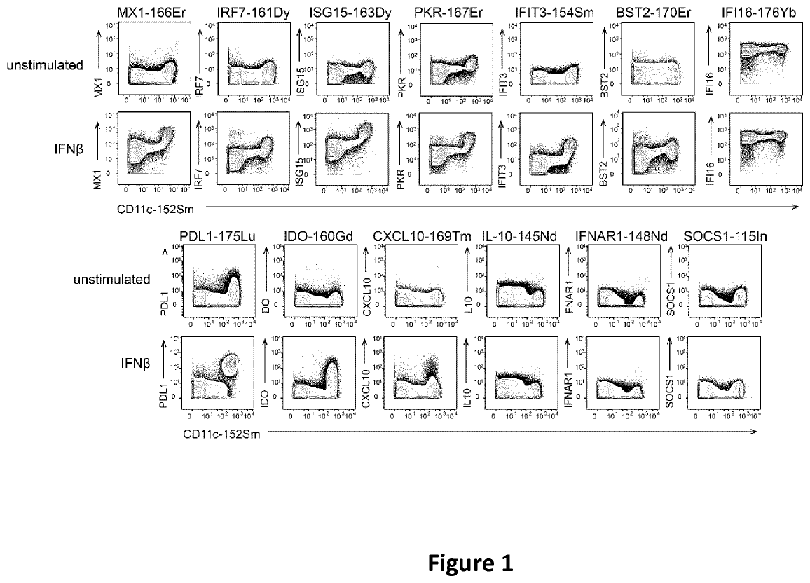

To interrogate ISG expression in single cells at the protein level, we developed a 41-parameter CyTOF panel for human proteins that simultaneously detects 13 ISGs, including anti-tumor/anti-pathogen (Mx1, PKR, IFIT3, IFI16, BST2, IFNαR1), stimulatory (IRF7, ISG15, CXCL10), and suppressive (IL-10, PDL1, IDO, SOCS1). We combined these with our validated CyTOF panel identifying immune cell populations and multiple transcriptional, functional, cytolytic, activating and inhibitory receptors¹¹ (Appendix). We validated the ISG panel by stimulating healthy donor PBMCs in vitro for 16 h in the presence of IFNβ or left them unstimulated. We observed that all ISGs except IFNAR1, IL10 and SOCS1 were further induced by IFN-I stimulation (FIG. 1), confirming the ability to detect and measure single-cell changes in ISG expression.

In an aspect, there is provided a method for predicting response to anti-PD1 based therapy in a subject with cancer, the method comprising: providing a sample of peripheral blood from the subject; adding an IFN-I to the sample; assessing T-cell response to IFN-I stimulation in the peripheral blood sample by measuring the expression of IFN-I stimulated genes; and predicting a better outcome in response to anti-PD1 therapy if the assessment in step c. indicates T-cell resistance to IFN-I stimulation and predicting a poorer outcome in response to anti-PD1 therapy if the assessment in step c. indicates T-cell responsiveness to IFN-I stimulation. In our study, we showed that the sensitivity of various immune cell subsets to IFN-Is can be used as a biomarker for predicting patient outcome in response to immunotherapy. While we narrowed in on 6 inflammatory ISGs in particular (BST2, IFIT3, IRF7, PKR, MX1, ISG15) as well as the induction of IDO1, the majority of genes controlled by IFN-Is would yield similar results given that upon stimulation these genes are largely regulated by the same downstream signaling events and are upregulated on a similar time-course at both the RNA and protein levels (Mostafavi et al. Cell 2016 and Megger et al. Frontiers in Immunology 2017). One study which exhaustively characterized the ISGs that are sensitive to IFN-I stimulation in both mouse and human found hundreds of common core ISGs upregulated across cell subsets and between species (Mostafavi et al. Cell 2016). Therefore, all ISGs that are upregulated after cells are exposed to IFN-Is, particularly the ones belonging to the major anti-viral families (IFIT, OAS, IFI, ISG, MX, STAT, IFITM, USP18) fall under the purview of this patent.

TABLE A
Markers with key lineage markers bolded

| | | | | | Used in |
|---|---|---|---|---|---|
| Metal tag | Specificity | Clone | Company | Studies | clustering |
| 89 Y | CD45 | HI30 | Fluidigm | All | No |
| 111Cd | CD80 | BB1 | BD Bioscience | All | No |
| 112Cd | CD45RO | UCHL1 | Biolegend | All | Yes |
| 115 In | SOCS1 | 4H1 | EMD Millipore | All | No |
| 116Cd | Ki67 | Ki-67 | Biolegend | All | No |
| 141 Pr | CD45RA | HI100 | Biolegend | All | Yes |
| 142 Nd | HLA-DR | L243 | Biolegend | All | Yes |
| 143 Nd | CD57 | HCD57 | Santa Cruz | All | Yes |
| | | | Biotech | | |
| 144 Nd | CD33 | WM53 | Biolegend | All | Yes |
| 145 Nd | anti-PE | PE001 | Fluidigm | combo | No |
| 145 Nd | IL-10 | JES3-9D7 | Biolegend | mono | No |
| 146 Nd | CD8a | RPA-T8 | Biolegend | All | Yes |
| 147 Sm | CD4 | RPA-T4 | Biolegend | All | Yes |
| 148 Nd | IFNAR1 | MMHAR-3 | pbl Bioscience | All | No |
| 149 Sm | FoxP3 | 236A-E7 | Thermofisher | All | Yes |
| 150 Nd | CD103 | B-Ly7 | Thermofisher | All | Yes |
| 151 Eu | CD39 | A1 | Biolegend | All | Yes |
| 152 Sm | CD11c | Bu15 | Biolegend | All | Yes |
| 153 Eu | CD3 | UCHT1 | Biolegend | All | Yes |
| 154 Sm | IFIT3 | 1G1 | Origene | All | No |
| 155 Gd | CD303 | 201A | Biolegend | All | Yes |
| 156 Gd | CD14 | M5E2 | Biolegend | All | Yes |
| 158 Gd | CD27 | O323 | Biolegend | All | No |
| 159 Tb | CD19 | HIB19 | Biolegend | All | Yes |
| 160 Gd | IDO | eyedio | Thermofisher | All | No |
| 161 Dy | IRF7 | 12G9A36 | Biolegend | All | No |
| 162 Dy | CD28 | CD28.2 | Biolegend | All | No |
| 163 Dy | ISG15 | 851707 | R & D Systems | All | No |
| 164 Dy | CD15 | W6D3 | Fluidigm | All | Yes |
| 165 Ho | PD1 | EH12.2H7 | Biolegend | All | No |
| 166 Er | Mx1 | ERP19967 | Abcam | All | No |
| 167 Er | PKR | 6H3A10 | Novus Biological | All | No |
| 168 Er | CXCR5 | MU5UBEE | Thermofisher | All | Yes |
| 169 Tm | CXCL10 | 4NYBUN | Thermofisher | All | No |
| 170 Er | BST2 | RS38E | Biolegend | All | No |
| 171 Yb | Granzyme B | GB11 | Thermofisher | All | Yes |
| 172 Yb | CD127 | eBioRDR5 | Thermofisher | All | Yes |
| 173 Yb | CD56 | HCD56 | Biolegend | All | Yes |
| 174 Yb | TCF-1 | S33-966 | BD Bioscience | All | Yes |
| 175 Lu | PDL1 | 29E.2A3 | Biolegend | All | No |
| 176 Yb | IFI16 | IG7 | Santa Cruz | All | No |
| | | | Biotech | | |
| 191 Ir | DNA (Cell ID) | | Fluidigm | All | No |
| 193 Ir | DNA (Cell ID) | | Fluidigm | All | No |
| 209 Bi | CD16 | 3G8 | Fluidigm | All | Yes |
| None | IL-10-PE | JES3-9D7 | Biolegend | combo | No |

The term “level of expression” or “expression level” as used herein refers to a measurable level of expression of the products of biomarkers, such as, without limitation, the level of messenger RNA transcript expressed or of a specific exon or other portion of a transcript, the level of proteins or portions thereof expressed of the biomarkers, the number or presence of DNA polymorphisms of the biomarkers, the enzymatic or other activities of the biomarkers, and the level of specific metabolites.

In addition, a person skilled in the art will appreciate that a number of methods can be used to determine the amount of a protein product of the biomarker of the invention, including immunoassays such as Western blots, ELISA, and immunoprecipitation followed by SDS-PAGE and immunocytochemistry.

As used herein, the term “control” refers to a specific value or dataset that can be used to prognose or classify the value e.g. expression level or reference expression profile obtained from the test sample associated with an outcome class. A person skilled in the art will appreciate that the comparison between the expression of the biomarkers in the test sample and the expression of the biomarkers in the control will depend on the control used.

The term “differentially expressed” or “differential expression” as used herein refers to a difference in the level of expression of the biomarkers that can be assayed by measuring the level of expression of the products of the biomarkers, such as the difference in level of messenger RNA transcript or a portion thereof expressed or of proteins expressed of the biomarkers. In a preferred embodiment, the difference is statistically significant. The term “difference in the level of expression” refers to an increase or decrease in the measurable expression level of a given biomarker, for example as measured by the amount of messenger RNA transcript and/or the amount of protein in a sample as compared with the measurable expression level of a given biomarker in a control.

The term “sample” as used herein refers to any fluid, cell or tissue sample from a subject that can be assayed for biomarker expression products and/or a reference expression profile, e.g. genes differentially expressed in subjects.

In some embodiments, the IFN-I stimulated genes comprise downstream components of IFN-I signaling.

In some embodiments, the IFN-I stimulated genes comprise MX1, PKR, IFIT3, BST2, IRF7, ISG15, and IDO1. Preferably, the IFN-I stimulated genes comprise MX1, PKR, IFIT3, IFI16, BST2, IFNAR1, IRF7, ISG15, CXCL10, IL10, PD-L1, IDO1, and SOCS1. Further preferably, the IFN-I stimulated genes consist of MX1, PKR, IFIT3, IFI16, BST2, IFNAR1, IRF7, ISG15, CXCL10, IL10, PD-L1, IDO1, and SOCS1.

In some embodiments, the measuring is performed using single-cell mass or flow cytometry.

In some embodiments, the measuring further comprises screening for phenotypic markers that distinguish between and among different immune cells types and other cells, preferably between T-cells, B-cells and myeloid cells. Preferably, the measuring further comprises screening for phenotypic markers that distinguish between naïve T-cells and effector T-cells.

In some embodiments, the phenotypic markers comprise some or all of CD45RO, CD45RA, HLA-DR, CD57, CD33, CD8a, CD4, CD39, CD11c, CD3, CD14, CD27, CD19, CD28, CD15, Granzyme B, CD127, and CD16.

In some embodiments, the measuring is performed using an antibody panel comprising the antibodies listed in Table B.

TABLE B
Antibody Panel (bolded: 6 ISGS used for signature)

| Metal tag | Specificity | Clone | Company |
|---|---|---|---|
| 89 Y | CD45 | HI30 | Fluidigm |
| 111Cd | CD80 | BB1 | BD Bioscience |
| 112Cd | CD45RO | UCHL1 | Biolegend |
| 115 In | SOCS1 | 4H1 | EMD Millipore |
| 116Cd | Ki67 | Ki-67 | Biolegend |
| 141 Pr | CD45RA | HI100 | Biolegend |
| 142 Nd | HLA-DR | L243 | Biolegend |
| 143 Nd | CD57 | HCD57 | Santa Cruz Biotech |
| 144 Nd | CD33 | WM53 | Biolegend |
| 145 Nd | anti-PE | PE001 | Fluidigm |
| 145 Nd | IL-10 | JES3-9D7 | Biolegend |
| 146 Nd | CD8a | RPA-T8 | Biolegend |
| 147 Sm | CD4 | RPA-T4 | Biolegend |
| 148 Nd | IFNAR1 | MMHAR-3 | pbl Bioscience |
| 149 Sm | FoxP3 | 236A-E7 | Thermofisher |
| 150 Nd | CD103 | B-Ly7 | Thermofisher |
| 151 Eu | CD39 | A1 | Biolegend |
| 152 Sm | CD11c | Bu15 | Biolegend |
| 153 Eu | CD3 | UCHT1 | Biolegend |
| 154 Sm | IFIT3 | 1G1 | Origene |
| 155 Gd | CD303 | 201A | Biolegend |
| 156 Gd | CD14 | M5E2 | Biolegend |
| 158 Gd | CD27 | O323 | Biolegend |
| 159 Tb | CD19 | HIB19 | Biolegend |
| 160 Gd | IDO | eyedio | Thermofisher |
| 161 Dy | IRF7 | 12G9A36 | Biolegend |
| 162 Dy | CD28 | CD28.2 | Biolegend |
| 163 Dy | ISG15 | 851707 | R & D Systems |
| 164 Dy | CD15 | W6D3 | Fluidigm |
| 165 Ho | PD1 | EH12.2H7 | Biolegend |
| 166 Er | Mx1 | ERP19967 | Abcam |
| 167 Er | PKR | 6H3A10 | Novus Biological |
| 168 Er | CXCR5 | MU5UBEE | Thermofisher |
| 169 Tm | CXCL10 | 4NYBUN | Thermofisher |
| 170 Er | BST2 | RS38E | Biolegend |
| 171 Yb | Granzyme B | GB11 | Thermofisher |
| 172 Yb | CD127 | eBioRDR5 | Thermofisher |
| 173 Yb | CD56 | HCD56 | Biolegend |
| 174 Yb | TCF-1 | S33-966 | BD Bioscience |
| 175 Lu | PDL1 | 29E.2A3 | Biolegend |
| 176 Yb | IFI16 | IG7 | Santa Cruz Biotech |
| 191 Ir | DNA (Cell ID) | | Fluidigm |
| 193 Ir | DNA (Cell ID) | | Fluidigm |
| 209 Bi | CD16 | 3G8 | Fluidigm |
| None | IL-10-PE | JES3-9D7 | Biolegend |

In some embodiments, the T-cell response is in CD4/CD8 effector T-cells.

In some embodiments, the method further comprises calculating an IFN-I Score based on the average change in expression of the IFN-I stimulated genes upon exposure to IFN-I. The IFN-I score is preferably the herein referenced IFN-I sensitivity score (ISS) or IFN-I response capacity (IRC).

Preferably, where an IFN-I Score higher than a predetermined cut-off IFN-I Score of a control population indicates responsiveness to IFN-I stimulation and an IFN-I Score lower than the predetermined cut-off IFN-I Score of the control population indicates resistance to IFN-I stimulation.

In a preferred embodiment, the cut-off IFN-I Score is predetermined using maximally-selected log-rank statistics to determine a cut off that will give the highest separation of the groups based on overall survival.

In some embodiments, the IFN-I Score is based on CD4 effector T-cells.

In some embodiments, the IFN-I Score is based on CD8 effector T-cells.

In some embodiments, the prediction is based on IFN-I Scores from both CD4 effector T-cells and a CD8 effector T-cells.

In some embodiments, the prediction is further based on IDO induction in CD14+ monocytes.

In some embodiments, the prediction is further based on an additional known biomarker, preferably PDL1 expression. In some embodiments, if the subject is predicted to have a better outcome in response to anti-PD1 therapy, then the method further comprises treating the subject with anti-PD1 therapy.

Preferably, treating the subject with anti-PD1 therapy comprises administering to the subject a therapeutically effective amount of Nivolumab, Pembrolizumab, Cemiplimab, Dostarlimab, Spartalizumab, Camrelizumab, Sintilimab, Tislelizumab, Toripalimab, JTX-4014, INCMGA00012, AMP-224, or AMP-514.

In some embodiments, if the subject is predicted to have a worse outcome in response to anti-PD1 therapy, then the method further comprises treating the subject with combination therapy comprising anti-PD1 therapy along with a further immunotherapy. In an embodiment, the further immunotherapy is preferably anti-CTLA4 therapy.

In an aspect, there is provided a method of treating a subject with cancer, the method comprising administering to the subject a therapeutically effective amount of a PD1 inhibitor, wherein the subject had been determined to have a better outcome in response to anti-PD1 therapy using the methods described herein.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

In some embodiments, the cancer is melanoma.

In some embodiments, the cancer is lung cancer.

In some embodiments, the cancer is head and neck cancer.

In some embodiments, the IFN-I is IFN-α, IFN-β, IFN-κ, IFN-δ, IFN-ε, IFN-τ, IFN-ω, or IFN-ζ.

In addition, the methods herein would be useful in clinical investigations and could assist in deciding who to include in clinical trials. A biomarker that is able to predict who would and would not respond to anti-PD1 immunotherapy could be very useful for inclusion criteria in clinical trials, particularly for anti-PD1 therapeutics.

As used herein, “pharmaceutically acceptable carrier” means any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent.

The advantages of the present invention are further illustrated by the following examples. The examples and their particular details set forth herein are presented for illustration only and should not be construed as a limitation on the claims of the present invention.

### EXAMPLES

### Methods and Materials

### Patients and Study Design

This study was conducted in accordance with the tenets of the Declaration of Helsinki and approved by the Research Ethics Board (REB) of the University Health Network. All donors provided written consent for sample collection.

Blood samples were obtained from melanoma (n=28) or NSCLC patients (n=34) undergoing standard-of-care anti-PD1. PBMCs were isolated and cryopreserved in liquid nitrogen. Pre-therapy samples were analyzed for all patients in this study.

### Sample Preparation and Stimulation

Viably frozen peripheral blood mononuclear cells (PBMCs) were thawed and counted. Cells were seeded in 24-well ultra-low attachment plates at a density of 1×10⁶ cells/ml. After a 1 hour recovery at 37° C., cells were left unstimulated in media or stimulated with 1000 U/ml IFN beta (IFNβ) for 16 h. Two hours before sample collection, brefeldin A and monensin were added to the cell culture. The same 2 to 3 healthy donor controls were used across all sets of experiments in order to evaluate batch variation.

### Mass Cytometry (CyTOF) Antibody Staining

Samples were stained with a panel of 38-41 surface and intracellular metal-tagged antibodies (Appendix), as described¹¹ with minor modifications to incorporate barcoding into the protocol. Briefly, cells were washed with PBS and samples were labelled with 1 μM natural abundance cis-platin (BioVision) for live/dead cell discrimination. Samples were fixed with Foxp3 Fixation/Permeabilization buffer (Thermofisher) for 10 min at room temperature and up to 20 samples were individually barcoded with the Cell-ID 20-Plex Pd Barcoding Kit (Fluidigm), then pooled for antibody labelling. Following Fc receptor blocking for 10 min at room temperate, the barcoded sample was incubated with a cocktail of surface antibodies for 30 min at 4° C. followed by washing and intracellular antibody staining for 30 min at 4° C. Finally, cells were labelled with 100 nM Cell-ID Intercalator-Ir (Fluidigm) for 1 hour at 4° C. to discriminate intact cells from debris and stored in PBS+1.6% PFA until acquisition for a maximum of 1 week.

### Bioinformatic Analyses

Data pre-processing and dimensionality reduction of CyTOF data: Preprocessing of files was performed using FlowJo 10 (v10.1r5) software. “fast-Phenograph” was run in R (v3.6.2) all other bioinformatic analysis was run in R (v3.5.3). Samples were manually debarcoded and exported as separated FCS files. Cells were filtered by gating on DNA, singlets, live cells and CD45+ cells. CD45+ raw signal events were exported as CSV files. CSV files were imported into R (v3.5.3) and randomly sampled to 1×10⁵ cells or 1.75×10⁵/file. All cells were included in dimensionality reduction for samples with fewer than the indicated cut-off. Marker expression values were arcsinh transformed using a custom co-factor for each marker. Phenograph and UMAP were run on each dataset separately (melanoma anti-PD-1 monotherapy, melanoma anti-PD-1/anti-CTLA-4 dual therapy and lung anti-PD-1 monotherapy) using the fast cluster algorithm from the package “fast-PG” and the R implementation of the “umap-learn” algorithm from the package “umap”. Clusters were then manually classified based on their lineage marker expression into the major immune cell categories.

IFN-I response capacity: The algorithm “scoreItems” from the R package, “psych” was used to compute the averages used as scores for IRC. IRC was calculated first by determining the change in expression between IFN-stimulated and unstimulated ISP expression. Because of this, data are first summarized by calculating the median expression of ISPs in the populations and conditions of interest. The change in expression was quantified as the arcsinh ratio (median stimulated-median unstimulated). The average arcsinh ratio of BST2, PKR, MX1, IFIT3, IRF7 and ISG15 represents the IRC. IFN-I sensitivity score (ISS) and IFN-I response capacity (IRC) are used interchangeably herein

Survival analysis: The R packages “survival” and “survminer” were used to fit and visualize the Kaplan-Meier estimates of overall survival as well as calculate the log-rank test. Cut-off estimates were calculated using the “surv_cutpoint” function from the “survminer” package with a minimum proportion setting of 0.3-0.5 to select cut-offs that not only give the best estimates but also that apply to a reasonable proportion of patients. Overall Survival months were calculated as the difference between the date of death and the date of C1. For censored data, Overall Survival months were calculated as the difference between the date the follow-up information was accessed and the date of C1. Progression-free survival time was calculated as the difference between date of progression and date of C1. For non-progressors, the date of last follow-up was used.

### Statistical Analysis

Statistical analysis was performed in R (v3.5.3). Survival analysis p-values were determined by the log-rank test.

### Results and Discussion

To interrogate ISG expression in single cells at the protein level, we developed a 41-parameter CyTOF panel for human proteins that simultaneously detects 13 ISGs, including anti-tumor/anti-pathogen (Mx1, PKR, IFIT3, IFI16, BST2, IFNαR1), stimulatory (IRF7, ISG15, CXCL10), and suppressive (IL-10, PDL1, IDO, SOCS1). We combined these with our validated CyTOF panel identifying immune cell populations and multiple transcriptional, functional, cytolytic, activating and inhibitory receptors¹¹ (Appendix). We validated the ISG panel by stimulating healthy donor PBMCs in vitro for 16 h in the presence of IFNβ or left them unstimulated. We observed that all ISGs except IFNAR1, IL10 and SOCS1 were further induced by IFN-I stimulation (FIG. 1), confirming the ability to detect and measure single-cell changes in ISG expression.

### Low Pre-Therapy Sensitivity to IFN-I Stimulation by Effector T Cell Subsets is Associated With Long-Term Survival to Anti-PD1 Immunotherapy

To investigate whether IFN-I responsiveness relates to clinical outcome, we devised a score to quantify the level of IFN-I induced ISG induction within a given cell subset by averaging the change in ISG protein expression between IFNβ vs unstimulated cells. A higher IFN-I response capcacity (IRC) indicates more ISGs upregulated to a greater extent. We ultimately focused on changes in the protein expression in a core set of 6 ISGs (BST2, PKR, ISG15, MX1, IFIT3, IRF7) identified as consistently sensitive to IFN-I induction by multiple immune cell subsets.

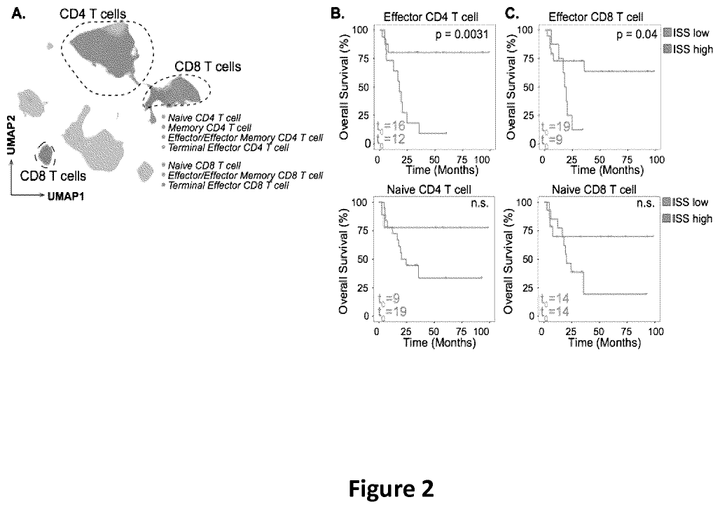

We analyzed a cohort of 28 cutaneous melanoma patients treated with anti-PD1 (Table 1, FIG. 2A). When patient cell populations of naïve (CD45RA⁺CD127⁺CD27⁺CD57⁻), effector/effector memory (CD45RA^{-/+}CD127^{+/-}CD27⁻CD39^{+/-}GzmB^{+/-}CD57⁻), or terminal effector (CD45RA^{-/+}CD127^{+/-}CD27⁻CD57⁺GzmB⁺) T cells were separated based on high and low ISS, a low ISS in effector CD4 T cells or effector CD8 T cells was strongly associated with OS (FIG. 2B, 2C). As noted previously, IFN-I sensitivity score (ISS) and IFN-I response capacity (IRC) are used interchangeably herein. In fact, for effector CD4 T cells, when the 11 patients were measured that had reached >2 years past the initiation of therapy at the time of writing, 8 patients (72.7%) with low ISS survived for longer than 2 years while only 3 patients (27%) with high ISS survived (FIG. 2B). The association was not observed when patients naïve CD4 T cells (FIG. 2B) or naïve CD8 T cells (FIG. 2C) were grouped based on high and low ISS.

TABLE 1
Melanoma Monotherapy Cohort

Characteristic

| | |
|---|---|
| Age, median (range) | 68 (31-85) |
| Gender, N (%) | |
| male | 19 (67.9%) |
| female | 9 (32.1%) |
| Diagnosis, N (%) | |
| Cutaneous Melanoma | 25 (89.2%) |
| Cutaneous Melanoma/Merkel Cell | 1 (3.6%) |
| Cutaneous Melanoma/Lung cancer | 1 (3.6%) |
| Cutaneous Melanoma/Lung adenocarcinoma/Breast cancer | 1 (3.6%) |
| Response (Medical Oncology), N (%) | |
| CR | 8 (28.6%) |
| PR | 9 (32.1%) |
| PR then PD | 1 (3.6%) |
| Mixed Response/SD | 3 (10.7%) |
| PD | 7 (25%) |
| Overall survival months, median | 19.97 |

### High IDO Induction is Associated With Longer Overall Survival After Anti-PD1 Therapy

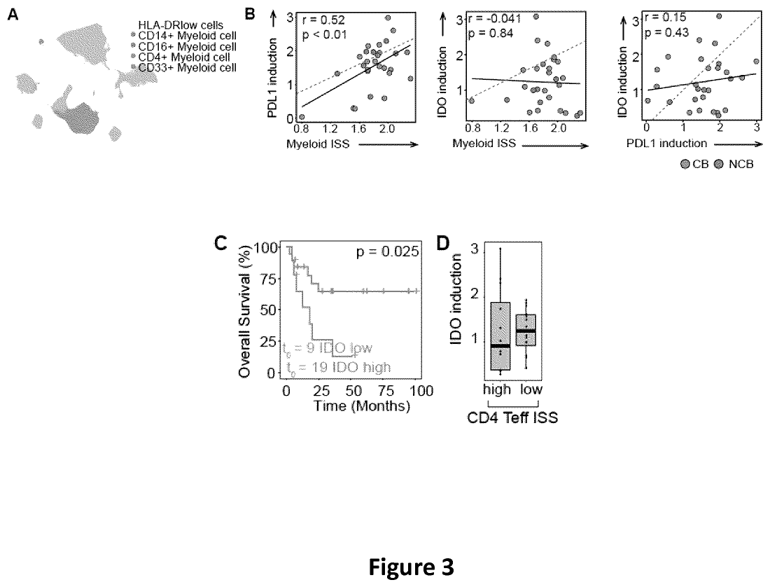

We next determined whether the upregulation of immunosuppressive ISGs PDL1 and IDO in response to IFN-I was related to patient outcome after anti-PD1 therapy. To investigate this, we focused on the myeloid compartment (FIG. 3A), since PDL1 and IDO induction are largely restricted to these cells. We noted that the induction of PDL1 accompanied the induction of the 6 inflammatory ISGs quantified by ISS, while IDO induction was not significantly correlated (FIG. 3B, left and middle panels). Furthermore, we noted an uncoupling between the induction of IDO and the induction of PDL1 across patients, suggesting that IDO and PDL1 are not co-regulated. To determine whether this uncoupling affects outcome, we stratified patients based on their ability to upregulate myeloid IDO expression in response to IFN-I and measured overall survival probability. Intriguingly, patients with higher induction of IDO had a significantly longer overall survival probability than patients with low IDO induction (FIG. 3C). This biomarker is independent of T cell ISS, since there was no significant difference in myeloid cell IDO induction between patients with high or low effector CD4 T cell IFN-I sensitivity (FIG. 3D). These data demonstrate that the induction of both inflammatory and immunosuppressive ISGs in response to IFN-I associate to patient outcome, and can be used as biomarkers to predict outcome.

### Use of Pre-Therapy Sensitivity to IFN-I for Treatment Selection

Combining anti-PD1 with other immunotherapies (e.g., anti-CTLA4) can increase survival in patients, but comes with a heightened risk of adverse events that require therapy cessation. Therefore, combination therapy is not suitable for every patient, and by contrast, some patients who do not respond to monotherapy may benefit from combination therapy. However there is currently no way to determine which therapy a patient should receive. Biomarkers that can facilitate treatment selection are urgently needed to address this problem. We propose that our technology can be used to select the optimal treatment for each patient. We have shown that patients with high IRC in specific T cell subsets do not benefit from anti-PD1 monotherapy. In this way, we can distinguish a patient that has a high IRC and therefore will not respond to anti-PD1 monotherapy, but would respond to combination therapy.

### Pre-Therapy IFN-I Sensitivity Predicts Progression-Free Survival in Lung Cancer Patients Bearing Squamous Cell Cancers or Adenocarcinomas That are p53 Mutated

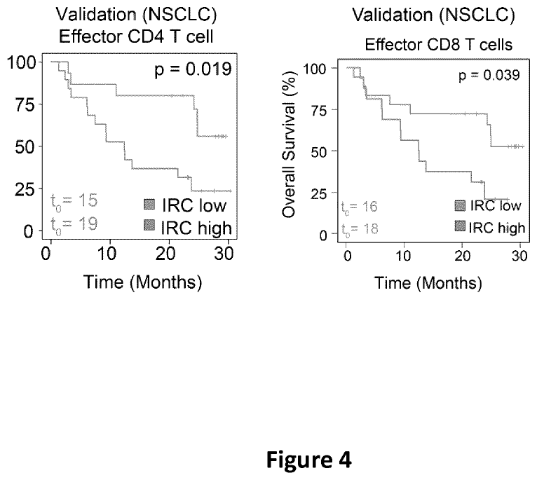

Finally, we wanted to determine whether low pre-therapy IFN-I sensitivity was associated with better treatment outcomes in other types of cancer. We analyzed pre-treatment PBMCs from a cohort of 34 lung cancer patients undergoing anti-PD1 therapy (Table 2), as before. The relationship between low IRC and longer OS was consistently observed in patients with NSCLC (FIG. 4). This was true for all the T cell subsets of interest (FIG. 4). Thus, IRC was a robust measurement to predict patient outcome in lung cancer as well, consistent with previous findings.

TABLE 2
Lung cancer anti-PD1 monotherapy cohort

| Characteristic | |
|---|---|
| Age, median (range) | 69 (53-90) |
| Gender, N (%) | |
| male | 17 (50%) |
| female | 17 (50%) |
| Diagnosis, N (%) | |
| Adenocarcinoma | 24 (70.5%) |
| Squamous cell carcinoma | 9 (26.5%) |
| Adenosquamous | 1 (2.9%) |
| Response, N (%) | |
| CR | 2 (5.9%) |
| PR | 12 (35.2%) |
| SD | 8 (23.5%) |
| PD | 12 (35.2%) |
| Line of treatment, N (%) | |
| 1 | 25 (73.5%) |
| 2 | 9 (26.4%) |
| Overall survival, median | 23.8 |

Taken together, this data from studies conducted across two cancer types and two treatment regimens presents strong evidence that low sensitivity of specific T cell subsets is associated with better outcomes in response to immunotherapy.

### Integrating Multiple Cellular Responses to IFN-I Improves the Prediction of Outcome

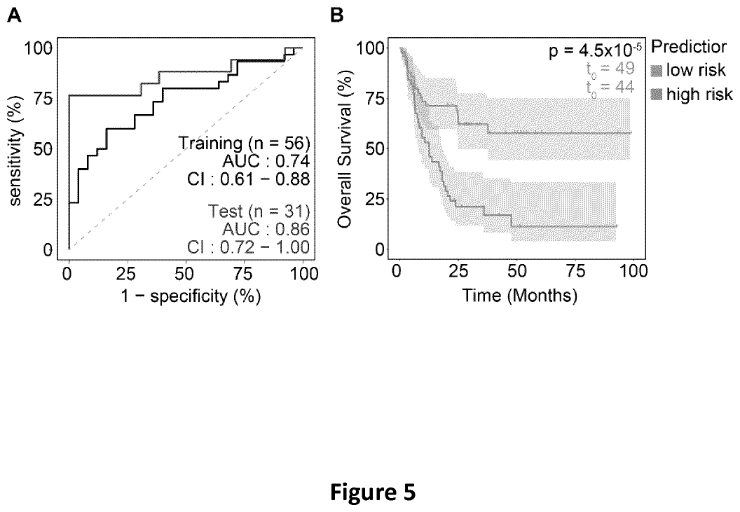

To better predict patient survival following PD1 blockade, we used a Cox proportional hazards model to integrate distinct IRC features, incorporating survival time. Patient samples grouped into a training set (n=59) and a test set (n=34) were used to construct and test the model, respectively. The features included were: CD4 and CD8 Teff cell IRC and IDO induction in CD14+ monocytes. The model could predict 2-year survival and overall survival with a high degree of accuracy in both the training and test sets (FIG. 5A) The model identified 81.5% (95% CI: 65.1-91.6%) of low risk patients surviving longer than 2 years (FIG. 5B). Further, the model correctly predicted 70.2% (95% CI: 54.9-82.2%) of patients progressing within 2 years and 65.3% (95% CI: 50.8%-77.6%) progressing within 3 years (FIG. 5B). Thus, integrating pre-therapy IRC features generated a predictive model of survival after therapy.

### Combining the Model Predictor With Other Biomarkers Strengthens the Predictive Power

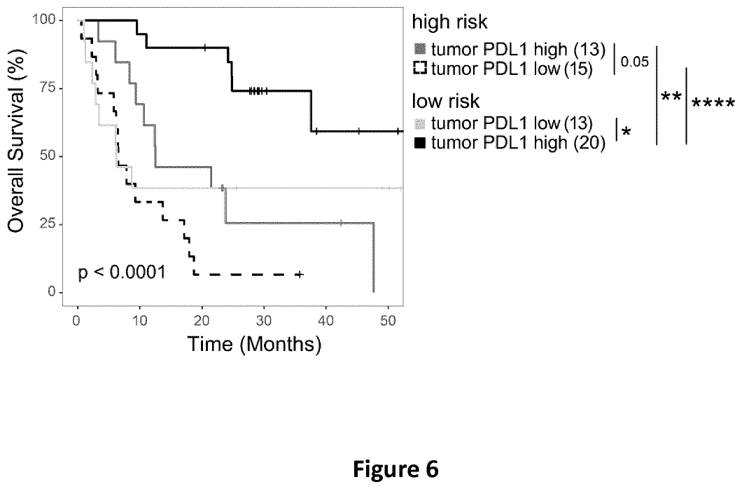

Other biomarkers have had limited and variable success in predicting patient outcomes, such as PDL1 expression in the tumor, (PMID: 31655605). As a result, despite an abundance of biomarker studies, few biomarkers are used clinically. It is possible that our novel predictor combined with existing biomarkers could further improve their reliability and performance. As a proof of principle, patients that we had previously identified as high or low risk (from FIG. 5) were further stratified based on their pre-therapy PDL1 expression in the tumor (measured by histology; n=61). Next, we analyzed overall survival of the resulting four groups and found that patients predicted to be at low risk of progression and with high PDL1 expression in the tumor survived significantly longer than any other group of patients (FIG. 6). Further, the patients predicted to be high risk with low expression of PDL1 had the worst outcome, with only 1 patient out of 15 surviving past 20 months (FIG. 6). This data supports combining our novel biomarker with existing biomarkers as a strategy to further stratify the high and low prediction designations to better identify the patients who will benefit from anti-PD1 immunotherapy.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCE LIST

1. Boukhaled, G.M., Harding, S. & Brooks, D.G. Opposing Roles of Type I Interferons in Cancer Immunity. Annu Rev Pathol 16, 167-198 (2021).
2. Wherry, E.J. & Kurachi, M. Molecular and cellular insights into T cell exhaustion. Nature Reviews Immunology 15, 486-499 (2015).
3. Wu, T. et al. The TCF1-Bcl6 axis counteracts type I interferon to repress exhaustion and maintain T cell stemness. Science Immunology 1, eaai8593 (2016).
4. Benci, J.L. et al. Opposing Functions of Interferon Coordinate Adaptive and Innate Immune Responses to Cancer Immune Checkpoint Blockade. Cell 178, 933-948.e914 (2019).
5. Benci, J.L. et al. Tumor Interferon Signaling Regulates a Multigenic Resistance Program to Immune Checkpoint Blockade. Cell 167, 1540-1554.e1512 (2016).
6. Uyttenhove, C. et al. Evidence for a tumoral immune resistance mechanism based on tryptophan degradation by indoleamine 2,3-dioxygenase. Nature medicine 9, 1269-1274 (2003).
7. Spranger, S. et al. Up-Regulation of PD-L1, IDO, and T<sub>regs</sub> in the Melanoma Tumor Microenvironment Is Driven by CD8<sup>+</sup> T Cells. Science Translational Medicine 5, 200ra116-200ra116 (2013).
8. Chen, J. et al. Type I IFN protects cancer cells from CD8+ T cell-mediated cytotoxicity after radiation. The Journal of clinical investigation 129, 4224-4238 (2019).
9. Lukhele, S., Boukhaled, G.M. & Brooks, D.G. Type I interferon signaling, regulation and gene stimulation in chronic virus infection. Seminars in Immunology 43, 101277 (2019).
10. Snell, L.M., McGaha, T.L. & Brooks, D.G. Type I Interferon in Chronic Virus Infection and Cancer. Trends Immunol 38, 542-557 (2017).
11. Gadalla, R. et al. Validation of CyTOF Against Flow Cytometry for Immunological Studies and Monitoring of Human Cancer Clinical Trials. Frontiers in Oncology 9 (2019).

1. A method for predicting response to anti-PD1 based therapy in a subject with cancer, the method comprising:
a. providing a sample of peripheral blood from the subject;
b. adding an IFN-I to the sample;
c. assessing T-cell response to IFN-I stimulation in the peripheral blood sample by measuring the expression of IFN-I stimulated genes;
d. predicting a better outcome in response to anti-PD1 therapy if the assessment in step c. indicates lower T-cell responsiveness (i.e., resistance) to IFN-I stimulation and predicting a poorer outcome in response to anti-PD1 therapy if the assessment in step c. indicates higher T-cell responsiveness to IFN-I stimulation.

2. The method of claim 1, wherein the IFN-I stimulated genes comprise downstream components of IFN-I signaling.

3. The method of claim 2, wherein the IFN-I stimulated genes comprise MX1, PKR, IFIT3, BST2, IRF7, ISG15, and IDO1; or comprise MX1, PKR, IFIT3, IFI16, BST2, IFNAR1, IRF7, ISG15, CXCL10, IL10, PD-L1, IDO1, and SOCS1; or consist of MX1, PKR, IFIT3, IFI16, BST2, IFNAR1, IRF7, ISG15, CXCL10, IL10, PD-L1, IDO1, and SOCS1.

4. (canceled)

5. (canceled)

6. The method of claim 1, wherein the measuring is performed using single-cell mass or flow cytometry.

7. The method of claim 1, wherein the measuring further comprises screening for phenotypic markers that distinguish between and among different immune cells types and other cells, preferably between T-cells, B-cells and myeloid cells.

8. The method of claim 7, wherein the measuring further comprises screening for phenotypic markers that distinguish between naïve T-cells and effector T-cells.

9. The method of claim 7, wherein the phenotypic markers comprise some or all of CD45RO, CD45RA, HLA-DR, CD57, CD33, CD8a, CD4, CD39, CD11c, CD3, CD14, CD27, CD19, CD28, CD15, Granzyme B, CD127, and CD16.

10. The method of claim 7, wherein the measuring is performed using an antibody panel comprising the antibodies listed in Table B.

11. The method of claim 1, wherein the T-cell response is in CD4/CD8 effector T-cells.

12. The method of claim 1, further comprising calculating an IFN-I Score based on the average change in expression of the IFN-I stimulated genes upon exposure to IFN-I.

13. The method of claim 12, wherein an IFN-I Score higher than a predetermined cut-off IFN-I Score of a control population indicates responsiveness to IFN-I stimulation and an IFN-I Score lower than the predetermined cut-off IFN-I Score of the control population indicates resistance to IFN-I stimulation.

14. The method of claim 13, wherein the cut-off IFN-I Score is predetermined using maximally-selected log-rank statistics to determine a cut off that will give the highest separation of the groups based on overall survival.

15. The method of claim 1, wherein the IFN-I Score is based on CD4 effector T-cells, CD8 effector T-cells or both CD4 effector T-cells and a CD8 effector T-cells.

16. (canceled)

17. (canceled)

18. The method of claim 17, wherein the prediction is further based on IDO induction in CD14+ monocytes.

19. The method of claim 1, wherein the prediction is further based on an additional known biomarker, preferably PDL1 expression.

20. The method of claim 1, wherein if the subject is predicted to have a better outcome in response to anti-PD1 therapy, then the method further comprises treating the subject with anti-PD1 therapy.

21. The method of claim 20, wherein treating the subject with anti-PD1 therapy comprises administering to the subject a therapeutically effective amount of Nivolumab, Pembrolizumab, Cemiplimab, Dostarlimab, Spartalizumab, Camrelizumab, Sintilimab, Tislelizumab, Toripalimab, JTX-4014, INCMGA00012, AMP-224, or AMP-514.

22. The method of claim 1, wherein if the subject is predicted to have a poorer outcome in response to anti-PD1 therapy, then the method further comprises treating the subject with combincation therapy comprising anti-PD1 therapy along with a further immunotherpary.

23. The method of claim 22, wherein the further immunotherapy is anti-CTLA4 therapy.

24. A method of treating a subject with cancer, the method comprising administering to the subject a therapeutically effective amount of a PD1 inhibitor, wherein the subject had been determined to have a better outcome in response to anti-PD1 therapy using the method of claim 1.

25. (canceled)

260 (canceled)

27. (canceled)

28. The method of claim 1, wherein the IFN-I is IFN-α, IFN-β, IFN-κ, IFN-δ, IFN-ε, IFN-τ, IFN-ω, or IFN-ζ.
